(19) United States
(12) Patent Application Publication
(10) Pub. No.: US 2016/0001265 A1
(43) Pub. Date: Jan. 7, 2016

(54) ALUMINA MATERIALS WITH INCREASED SURFACE ACIDITY, METHODS FOR MAKING, AND METHODS FOR USING THE SAME

(71) Applicant: UOP LLC, Des Plaines, IL (US)

(72) Inventors: Richard R. Willis, Cary, IL (US); Colleen Costello, Chicago, IL (US); Jeffrey Noga, Woodbridge, IL (US); Paulina Jakubczak, Elk Grove Village, IL (US)

(21) Appl. No.: 14/321,657

(22) Filed: Jul. 1, 2014

(51) Int. Cl. B01J 21/04; C01F 7/02; B01J 35/10; C07C 4/06

(52) U.S. Cl. CPC B01J 21/04; C07C 4/06; C01F 7/02; B01J 35/1019; C07C 2521/04

(57) ABSTRACT

Aluminas with increased surface acidity, methods of making the same, and methods for using the same are provided. In an exemplary embodiment, a method for increasing the surface acidity of an alumina material includes providing an alumina starting material, and processing the alumina starting material under hydrothermal conditions in the presence of one or more organic acids to generate a hydrothermally treated alumina. In this embodiment, the one or more organic acids includes a polyprotic organic acid with a pKa value of about 0 to about 10, and the resulting hydrothermally treated alumina has increased surface acidity relative to the alumina starting material.

### TECHNICAL FIELD

The technical field generally relates to alumina materials, methods for making the same, and methods for using the same. More particularly, the technical field relates to hydrothermally treated aluminas with increased surface acidity, methods for making the same, and methods for using the same.

### BACKGROUND

Gamma alumina, or gamma aluminum (III) oxide, is widely used as a catalyst support for many important industrial catalyzed reactions. For instance, gamma alumina is commonly used as a support material for hydrotreating and hydrocracking catalysts in the petroleum products industry. Gamma alumina owes its widespread use to several factors, including its low cost, mechanical strength, high surface area, and large volume of open mesoporosity.

When employed as a catalyst support, several characteristics of gamma-aluminas, including crystal size and morphology, surface area and surface area stability, and pore size distribution, impact catalytic behavior of the supported catalysts. One characteristic of particular importance is surface acidity, which can impact total conversion efficiency of the supported catalyst.

Accordingly, it is desirable to provide novel gamma aluminas that are suitable for use as catalyst supports with desirable improved surface acidity characteristics, as well as methods for making and using the same. Furthermore, other desirable features and characteristics will become apparent from the subsequent detailed description and the appended claims, taken in conjunction with the accompanying drawings and the foregoing technical field and background.

### BRIEF SUMMARY

Aluminas with increased surface acidity, methods of making the same, and methods for using the same are provided. In one embodiment, a method for increasing surface acidity of an alumina material is provided. One such method includes providing an alumina starting material; and processing the alumina starting material under hydrothermal conditions in the presence of one or more organic acids to generate a hydrothermally treated alumina. The resulting hydrothermally treated alumina has increased surface acidity relative to the alumina starting material. In some embodiments, the one or more organic acids include a polyprotic organic acid with a pKa value of about 0 to about 10.

In another embodiment, a catalyst capable of catalyzing the conversion of 1-heptene to C3 and C4 is provided. In this embodiment, the catalyst comprises an alumina that has been hydrothermally treated in the presence of an organic acid. When a 250 cc/min stream of 1-heptene is contacted with the catalyst at a temperature of about 425° C., the total 1-heptene conversion is about 60% or more.

In another embodiment, a method for the catalytic conversion of 1-heptene is provided. In this embodiment, a feed stream comprising 1-heptene is provided and contacted with a catalyst comprising an alumina that has been hydrothermally treated in the presence of an organic acid. The catalytic conversion generates a product stream comprising one or more catalytically generated constituents; wherein when a 250 cc/min feed stream of 1-heptene is contacted with the catalyst at a temperature of about 425° C., the total 1-heptene conversion is about 50% or more.

### DETAILED DESCRIPTION

The following detailed description is merely exemplary in nature and is not intended to limit the exemplary methods, compositions, or systems described herein. Furthermore, there is no intention to be bound by any theory presented in the preceding background or the following detailed description.

Aluminas with increased surface acidity, methods of making, and methods and systems for using the same are described herein. Hydrotreating or hydrocracking catalysts supported by such aluminas show increased catalytic activity relative to supported catalysts with relatively reduced surface acidity. Further, aluminas prepared according to methods provided herein demonstrate improved surface area stability.

Methods described herein provide a synthesis route for aluminas with increased surface activity via hydrothermal treatment in the presence of one or more organic acids.

Specifically, in some embodiments, gamma alumina is converted to boehmite under hydrothermal treatment conditions in the presence of one or more organic acids.

In some embodiments, an organic acid used in a hydrothermal treatment process is a polyprotic organic acid. As used herein, a polyprotic organic acid is an organic acid that is able to donate more than one proton per acid molecule. Such acids include organic acids with a plurality of carboxylic acid groups per molecule. In some embodiments, an organic acid used in a hydrothermal treatment process is an organic acid with a pKa value of about 0 to about 10. Such organic acids may be referred to herein as complexing acids. In some embodiments, an organic acid used in a hydrothermal treatment process is tartaric acid, malic acid, citric acid, or a mixture thereof.

Various hydrothermal processing conditions may be employed in the methods described herein. It is known in the art that gamma alumina may be hydrated and at least partially converted to boehmite by hydrothermal treatment. See, e.g., U.S. Pat. No. 7,402,612. Thus, in some embodiments, hydrothermal processing conditions include subjecting a mixture of gamma alumina and a hydrothermal treatment solution to an elevated temperature for a sufficient amount of time to convert at least a portion of the gamma alumina to boehmite In some embodiments, hydrothermal processing conditions include subjecting a mixture of gamma alumina and a hydrothermal treatment solution to an elevated temperature for a sufficient amount of time to convert substantially all of the gamma alumina is converted to boehmite during hydrothermal treatment. In some embodiments, hydrothermal processing conditions include subjecting a mixture of gamma alumina and a hydrothermal treatment solution at a ratio of about 0.5:1 to about 1:0.5, such as about 1:1, to an elevated temperature for a sufficient amount of time to convert at least a portion of the gamma alumina to boehmite In these embodiments, the hydrothermal treatment solution comprises water and one or more suitable organic acids, such one or more organic acids meeting the conditions provided above. It will be understood that the extent of conversion depends on both the time and temperature of hydrothermal processing. For instance, less time is necessary at higher temperatures to substantially complete the conversion, while lower temperatures require more time to reach the same extent of conversion. In some embodiments, the hydrothermal processing conditions include subjecting a mixture of gamma alumina and hydrothermal treatment solution to a temperature of about 100° C. to about 300° C., such as about 100° C. to about 250° C., such as about 150° C. to about 200° C., for a time sufficient to convert at least a portion of the gamma alumina to boehmite In some embodiments, the hydrothermal processing conditions include subjecting a mixture of gamma alumina and hydrothermal treatment solution to a temperature sufficiently high to convert at least a portion of the gamma alumina to boehmite for a period of time of at least about 2 hours, such as at least about 4 hours, such as at least about 6 hours.

As used herein, the term “substantially all” when used to describe the extent of a reaction or purity of a composition, means that unreacted components or impurities in a composition may be present but at a level which does not impact a physical or chemical characteristic of the composition in a meaningful way. Quantitatively, “substantially all” indicates about 90% or more, such as about 95% or more, such as about 97.5% or more, such as about 99% or more.

In embodiments, the amount of one or more organic acids initially present in the hydrothermal treatment solution may vary. In some embodiments, the hydrothermal treatment solution initially comprises from about 0.5 wt. % to about 25 wt. %, such as about 1 wt. % to about 20 wt. %, such as about 1 wt. % to about 15 wt. %, organic acids, relative to the gamma alumina on a volatile free basis. In some embodiments, the hydrothermal treatment solution initially comprises from about 0.5 wt. % to about 25 wt. %, such as about 0.75 wt. % to about 15 wt. %, such as about 1 wt. % to about 10 wt. % tartaric acid, relative to the gamma alumina on a volatile free basis. In some embodiments, the hydrothermal treatment solution initially comprises from about 0.5 wt. % to about 25 wt. %, such as about 1 wt. % to about 15 wt. %, such as about 2 wt. % to about 10 wt. % malic acid, relative to the gamma alumina on a volatile free basis. In some embodiments, the hydrothermal treatment solution initially comprises from about 0.5 wt. % to about 25 wt. %, such as about 1 wt. % to about 15 wt. %, such as about 2 wt. % to about 10 wt. % citric acid, relative to the gamma alumina on a volatile free basis.

In some embodiments, the amount of carbon species present in the hydrothermal treatment solution is significantly reduced as hydrothermal treatment progresses. For instance, in an embodiment where the hydrothermal treatment solution initially contains about 1 wt. % tartaric acid relative to the gamma alumina on a volatile free basis, the amount of carbon species in the post-treatment hydrothermal treatment solution may be substantially undetectable (such as via NMR) after as little as about 3.5 hours of hydrothermal treatment. This means that in some embodiments substantially all of the one or more organic acids adsorb and/or react with the alumina during hydrothermal treatment. Of course, the extent of organic acid adsorption into and/or reaction with the alumina will vary with initial organic acid concentration, ratio of alumina to hydrothermal treatment solution, and the particular hydrothermal processing conditions (including time and temperature). In some embodiments, these conditions are selected such that at least about 50%, such as at least about 75%, such as at least about 90%, such as substantially all of the organic acid content originally present in the hydrothermal treatment solution is adsorbed and/or reacted with the alumina during hydrothermal treatment.

Further, in some embodiments, the amount of aluminum species present in the hydrothermal treatment solution does not significantly change as hydrothermal treatment progresses. For instance, in an embodiment where the hydrothermal treatment solution initially contains about 1% tartaric acid relative to the gamma alumina on a volatile free basis, the amount of aluminum species in the post-treatment hydrothermal treatment solution may be substantially undetectable via NMR or ICP after about 3.5 hours of hydrothermal treatment. This means that in some embodiments substantially no aluminum is leaching into the hydrothermal treatment solution from the alumina during hydrothermal treatment processing.

As used herein, the term “substantially undetectable” should be understood to mean that the analyte in question may be present in the substance being tested, but is present at an amount below the threshold of detectability for the test being used. Such limits of detection are readily ascertained by those of skill in the art. As an example, aluminum in an aqueous media may be substantially undetectable via ICP at levels of less than about 0.5 ppm.

In some embodiments the total organic content of boehmites generated via hydrothermal treatment methods provided herein increases relative to the total organic content of the gamma alumina starting material. Without wishing to be bound by theory, it is believed that this is due to adsorption of at least a portion of the one or more organic acids from the hydrothermal treatment solution, or adsorption of reaction products from the one or more organic acids and the surface of the alumina. For instance, in an embodiment where the hydrothermal treatment solution initially contains about 1 wt. % tartaric acid relative to the gamma alumina on a volatile free basis, the total organic content remaining in the hydrothermal treatment solution after about 24 hours of hydrothermal treatment may be less than about 50 ppm, such as less than about 25 ppm, such as less than about 20 ppm, or from about 10 ppm to about 50 ppm, such as from about 10 ppm to about 25 ppm, such as from about 10 ppm to about 20 ppm. In another exemplary embodiment where the hydrothermal treatment solution initially contains about 7.5 wt. % tartaric acid relative to the gamma alumina on a volatile free basis, the total organic content remaining in the hydrothermal treatment solution after about 3.5 hours of hydrothermal treatment may be less than about 100 ppm, such as less than about 75 ppm, such as less than about 50 ppm, or from about 25 ppm to about 100 ppm, such as from about 25 ppm to about 75 ppm, or from about 25 ppm to about 50 ppm. In a similar exemplary embodiment where the hydrothermal treatment solution initially contains about 7.5 wt. % tartaric acid relative to the gamma alumina on a volatile free basis, the total organic content remaining in the hydrothermal treatment solution after about 24 hours of hydrothermal treatment may be less than about 500 ppm, such as less than about 400 ppm, such as less than about 250 ppm, or from about 100 ppm to about 500 ppm, such as from about 100 ppm to about 400 ppm, or from about 100 ppm to about 250 ppm. In these exemplary embodiments, the total organic content of the resulting boehmite is about 1 wt. % to about 3 wt. % based on the weight of the dried boehmite

Upon conversion of gamma alumina to boehmite via conventional hydrothermal treatment (i.e., in the absence of an organic acid used in the methods provided herein), crystallite size increases. See, e.g., Souza Santos, P., Coelho, A.C.V., Souza Santos, H., Kiyohara, P. K. Mater. Res. 2009, 12, 437-445. Further, it is known that inclusion of certain acidic or basic components in the hydrothermal treatment solution affects particle morphology (e.g., needle-shaped, elliptical, platelet-shaped, near-spherical, etc.). See, e.g., U.S. Pat. No. 8,088,355. However, significant growth in crystallite size is observed with such treatments, and generally increases with increasing temperature and with increasing processing time.

It has been found that when one or more polyprotic organic acids, such as a polyprotic organic acid with a pKa value of about 0 to about 10, are included in the hydrothermal treatment solution as per the methods provided herein, crystallite size growth is significantly inhibited during hydrothermal conversion of gamma alumina to boehmite Inhibition of crystallite size growth is desirable at least for the reason that an increase in crystallite size typically correlates with a decrease with surface area High surface area is desirable for aluminas used as catalyst support materials as catalyst support materials with increased surface area exhibit improved mass transfer properties due to corresponding increased pore volume. Catalysts using such support materials tend to exhibit increased effectiveness, and thus are more cost efficient. In some particular embodiments, boehmite aluminas prepared according to organic acid—hydrothermal treatments described herein have an average crystallite size of less than about 60 Å, such as about 30 Å to about 50 Å, such as about 35 Å to about 45 Å.

In some embodiments, the methods further include calcining the hydrothermally derived boehmite material described above. Calcining a hydrothermally derived boehmite at an appropriate temperature and for a sufficient amount of time results in regeneration of a gamma alumina. Regenerated gamma aluminas prepared from boehmites generated from hydrothermal treatments described herein have increased surface acidity relative to the gamma alumina starting material. Further, due to an inhibitory effect of the one or more organic acids on crystal size growth, the regenerated gamma aluminas have surface areas similar to the surface areas of the starting gamma aluminas. For instance, in some embodiments, regenerated gamma aluminas prepared as described herein have Brunauer, Emmett and Teller (or BET) surface areas that are ±25%, such as ±10%, such as ±5%, such as ±3%, of the BET surface areas of the starting gamma aluminas. As such, surface areas of regenerated gamma aluminas prepared via methods similar to those described herein (i.e., conversion of gamma alumina to boehmite via hydrothermal treatment in the presence of one or more organic acids, followed by regeneration of gamma alumina via calcining the boehmite) differ significantly from surface areas of regenerated gamma aluminas similarly prepared but excluding organic acids from the hydrothermal treatment solution. For instance, regenerated gamma aluminas prepared without the one or more organic acids in the hydrothermal treatment solution have BET surface areas that may be reduced by as much as about 50% of the BET surface areas of the starting gamma aluminas.

Thus, in some embodiments, regenerated gamma aluminas have a combination of small crystallite size and high surface area. For instance, in some embodiments, regenerated gamma aluminas have an average crystallite size of less than about 60 Å, such as about 30 Å to about 50 Å, such as about 35 Å to about 45 Å, and a BET surface area of greater than about 125 m²/g, such as greater than about 175 m²/g or more, such as about 200 m²/g to about 300 m²/g.

It has further been found that the surface area stability of regenerated gamma aluminas prepared as described herein is also significantly improved relative to the starting gamma aluminas. In this regard, it has been observed that when a gamma alumina is subjected to steam calcination (i.e., calcining in the presence of water vapor), the surface area of the gamma alumina decreases. However, as with the inhibition of crystal growth observed during hydrothermal treatment, the presence of one or more organic acids inhibits this decrease in surface area. For instance, in some embodiments, regenerated gamma aluminas prepared as described herein exhibit about 40% drop in BET surface area or less when subjected to 40% steam calcining at 650° C. for about 6 hours. A decrease in BET surface area of about 40% or less a significant improvement over the about 60% or more decrease observed for regenerated gamma aluminas prepared via hydrothermal treatment and subsequent calcining, without inclusion of the one or more organic acids in the hydrothermal treatment solution.

Accordingly, in another aspect, boehmite and regenerated gamma aluminas prepared via hydrothermal treatment as described above are provided. These aluminas may have any combination of the above described characteristics, without limit In particular, boehmite and regenerated gamma aluminas are provided with increased surface acidity that may find use as adsorbents, catalyst, or as supports for other various conventional catalytic materials. For example, in some embodiments, a catalyst comprising a boehmite and regenerated gamma alumina as provided herein for the catalytic conversion of 1-heptene may exhibit an increase in catalytic activity of at least about 15% under conventional conditions (e.g., at about 425° C. and about 250 cc/min feed rate). When used under conventional conditions, catalysts comprising hydrothermally treated aluminas as provided herein may exhibit total 1-heptene conversion of at least about 40%, such as at least about 50%, such as about 40% to about 60%, such as about 50% to about 60%.

Thus, in yet another aspect, boehmite and regenerated gamma alumina catalysts and catalyst supports are provided herein. As indicated above, aluminas currently find widespread use in the art as supports for various catalysts, including hydrotreating and hydrocracking catalysts used in the petroleum processing industry. In some embodiments, boehmite and regenerated gamma alumina catalysts are provided. Such catalysts may include a boehmite and regenerated gamma alumina material as provided herein, and optionally any suitable catalytic material embedded or adsorbed therein according to conventional supported catalyst practice. For instance, supported catalysts may comprise low levels, e.g. <0.5%, of precious metals, such as platinum, or higher levels, e.g. >10%, of base metals such as molybdenum or tungsten. In an embodiment, a supported catalyst is provided herein that comprises a catalyst support comprising boehmite material prepared via hydrothermal treatment in the presence of one or more organic acids, and a nickel (Ni)-tungsten (W) catalytic material.

Preparation of catalysts or supported catalysts based on a boehmite or regenerated gamma alumina material as provided herein may be conducted via any conventional technique. For instance, a boehmite or regenerated gamma alumina may be prepared as provided herein, mixed with a suitable liquid carrier and optionally a desired catalytically active material to form a paste, extruded in any desired shape or form, and dried. Suitable liquid carriers and optional catalytically active materials and may be selected according to conventional practice by those of skill in the art.

It has been determined that the increase in surface acidity in boehmite and regenerated gamma alumina materials prepared via hydrothermal treatment in the presence of one or more organic acids, as provided herein, results in an improvement in the catalytic behavior of supported catalysts made therefrom. For instance, in some embodiments, catalysts and supported catalysts comprising boehmite and regenerated gamma alumina materials prepared via hydrothermal treatment in the presence of one or more organic acids as provided above exhibit improved catalytic activity. As used herein, catalytic activity is reflected in an amount of product(s) generated from a feed relative to the theoretical amount of product(s) that would be generated if 100% of the same feed were reacted. Generally, reactions catalyzed via alumina-supported catalysts exhibit increasing catalytic activity with increasing temperature. Thus, a difference in catalytic activity between two different supported catalysts may be expressed as the temperature difference necessary for both supported catalysts to yield the same amount of product(s) from the same feed.

In some embodiments, a catalyst comprises a modified boehmite prepared from an alumina starting material according to methods provided herein, silica alumina, nickel and tungsten. In some particular embodiments, the catalyst comprises about 1:1 silica alumina : modified boehmite In some embodiments, the catalyst comprises about 2 wt. % nickel, relative to the total weight of the catalyst. In some embodiments, the catalyst comprises about 20 wt. % tungsten, relative to the total weight of the catalyst.

In some embodiments, a catalyst comprising a modified alumina provided herein may be used to catalyze 1-heptene cracking to C3 and C4. In some related embodiments, the catalysts exhibit at least about 1° F. (0.556° C.), such as about 1° F. (0.556° C.) to about 5.0° F. (2.78° C.), such as about 2.0° F. (1.11° C.) to about 5.0° F. (2.78° C.), such as about 2.5° F. (1.39° C.) to about 5.0° F. (2.78° C.), such as about 2.5° F. (1.39° C.), increase in catalytic activity relative to the alumina starting material in place of the modified alumina.

Thus, in another aspect, methods of catalyzing a reaction are provided. In these methods, a feed stream comprising a component capable of undergoing a catalyzed reaction is contacted with a catalyst comprising modified boehmite prepared from an alumina starting material according to methods provided herein. In some embodiments, the catalyst comprises a catalytically active material and a support material comprising a modified boehmite prepared from an alumina starting material according to methods provided herein. In these embodiments, the catalytically active material is selected according to the particular reaction to be catalyzed. For instance, the catalyst may be a hydrotreating and hydrocracking catalyst that comprises a conventional catalyst material selected based on the identity of the component in the feed stream to be hydrotreated and/or hydrocracked.

Use of the aluminas described herein as catalyst support materials is not intended to be limited to support of any particular additional catalytically active material or to be limited to use in catalyzing any particular reaction. The following exemplary embodiment is provided for illustration purposes only. In this embodiment, a feed stream comprising 1-heptene is contacted with a catalyst comprising a modified boehmite prepared from an alumina starting material according to methods provided herein. Upon contact of 1-heptene from the feed stream with the catalyst under suitable conditions, heptene is catalytically converted resulting in generation of a product stream comprising C3 and C4. This catalytic reaction is generally known in the art and may be conducted under conventional conditions, including contacting the feed stream with the catalyst at a reaction temperature of about 400° C. to about 500° C. and at any suitable flow rate.

Those having skill in the art, with the knowledge gained from the present disclosure, will recognize that various changes could be made in the methods described herein without departing from the scope of the present invention. Mechanisms used to explain theoretical or observed phenomena or results, shall be interpreted as illustrative only and not limiting in any way the scope of the appended claims.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

What is claimed is:

1. A method for increasing surface acidity of an alumina material, the method comprising the steps of:
providing an alumina starting material; and
processing the alumina starting material under hydrothermal conditions in the presence of an organic acid to generate a hydrothermally treated alumina,
wherein the organic acid comprises a polyprotic organic acid with a pKa value of about 0 to about 10, and the hydrothermally treated alumina has increased surface acidity relative to the alumina starting material.

2. The method of claim 1, wherein the organic acids comprises tartaric acid, malic acid, citric acid, or a mixture thereof.

3. The method of claim 1, wherein the alumina starting material comprises a gamma alumina.

4. The method of claim 1, wherein the hydrothermally treated alumina comprises a boehmite alumina.

5. The method of claim 4, further comprising calcining the hydrothermally treated alumina to convert at least a portion of the boehmite alumina in the hydrothermally treated alumina into a gamma alumina.

6. The method of claim 5, wherein the gamma alumina has a Brunauer, Emmett and Teller (BET) surface area that is ±25% of the alumina starting material.

7. The method of claim 4, wherein substantially all of the hydrothermally treated alumina is a boehmite alumina.

8. The method of claim 7, further comprising calcining the hydrothermally treated alumina to convert substantially all of the hydrothermally treated alumina into a gamma alumina.

9. The method of claim 1, wherein processing the alumina starting material under hydrothermal conditions comprises subjecting a mixture of the alumina starting material and a hydrothermal treatment solution to an elevated temperature for a sufficient period of time to convert at least a portion of the alumina starting material to a boehmite alumina, wherein the alumina starting material and the hydrothermal treatment solution are present in the mixture at a ratio of about 0.5:1 to about 1:0.5.

10. The method of claim 9, wherein the elevated temperature is about 100° C. to about 300° C.

11. The method of claim 9, wherein the period of time is at least about 2 hours.

12. The method of claim 9, wherein the hydrothermal treatment solution initially comprises about 0.5 wt. % to about 25 wt. % one or more organic acids relative to the weight of the gamma alumina on a volatile free basis.

13. The method of claim 9, wherein the hydrothermal treatment solution initially comprises about 0.5 wt. % to about 25 wt. % tartaric acid relative to the weight of the gamma alumina on a volatile free basis.

14. The method of claim 9, wherein the hydrothermal treatment solution initially comprises about 0.5 wt. % to about 25 wt. % malic acid relative to the weight of the gamma alumina on a volatile free basis.

15. The method of claim 9, wherein the hydrothermal treatment solution initially comprises about 0.5 wt. % to about 25 wt. % citric acid relative to the weight of the gamma alumina on a volatile free basis.

16. A catalyst capable of catalyzing the conversion of 1-heptene to C3 and C4, the catalyst comprising an alumina that has been hydrothermally treated in the presence of an organic acid, wherein when a 250 cc/min stream of 1-heptene is contacted with the catalyst at a temperature of about 425° C., the total 1-heptene conversion is about 50% or more.

17. The composition of claim 16, wherein the hydrothermally treated alumina comprises a boehmite alumina.

18. The composition of claim 16, wherein the hydrothermally treated alumina comprises a gamma alumina.

19. The composition of claim 16, wherein the hydrothermally treated alumina has an average crystallite size of less than about 60 Å and a BET surface area of greater than about 125 m²/g.

20. A method for the catalytic conversion of 1-heptene, said method comprising:
providing a feed stream comprising 1-heptene;
contacting the feed stream with a catalyst comprising an alumina that has been hydrothermally treated in the presence of an organic acid; and
generating a product stream comprising one or more catalytically generated constituents; wherein when a 250 cc/min feed stream of 1-heptene is contacted with the catalyst at a temperature of about 425° C., the total 1-heptene conversion is about 50% or more.
